(19) United States
(12) Patent Application Publication
(10) Pub. No.: US 2026/0232657 A1
(43) Pub. Date: Aug. 13, 2026

(54) MELANOCORTIN SUBTYPE-2 RECEPTOR (MC2R) ANTAGONIST FOR THE TREATMENT OF ACTH-DEPENDENT CUSHING'S SYNDROME

(71) Applicant: CRINETICS PHARMACEUTICALS, INC., San Diego, CA (US)

(72) Inventors: Peter TRAINER, Edinburgh (GB); Alan S. KRASNER, San Diego, CA (US); Sha LUO, San Diego, CA (US)

(21) Appl. No.: 19/471,865

(22) PCT Filed: Apr. 3, 2024
(86) PCT No.: PCT/US2024/022738

§ 371 Date: Oct. 2, 2025

Related U.S. Application Data
(60) Provisional application No. 63/457,144, filed on Apr. 5, 2023.

(51) Int. Cl. A61K 31/496; A61P 5/08; A61P 5/38

(52) U.S. Cl. CPC A61K 31/496; A61P 5/08; A61P 5/38

(57) ABSTRACT

Provided herein are methods and compositions for the treatment of ACTH-dependent Cushing's syndrome.

### CROSS-REFERENCE TO RELATED APPLICATIONS

This application claims the benefit of U.S. Provisional Patent Application No. 63/457,144, filed Apr. 5, 2023, which is incorporated herein in its entirety by reference.

### FIELD

Described herein is a melanocortin subtype-2 receptor (MC2R) antagonist and methods of using the MC2R antagonist in the treatment of ACTH-dependent Cushing's syndrome, which can be the result of a pituitary tumor (Cushing's disease) or an extra-pituitary tumor (ectopic ACTH syndrome).

### BACKGROUND

The melanocortin receptors form a family of G protein-coupled receptor (GPCRs) (MC1R, MC2R, MC3R, MC4R, and MC5R) that are selectively activated by different melanocortin peptides, adrenocorticotropic hormone (ACTH), and the melanocortin peptides α-, β-, and γ-melanocyte-stimulating hormone (α-MSH, β-MSH, and γ-MSH), that are all derived proteolytically from proopiomelanocortin hormone, or POMC. ACTH is a 39 amino acid peptide that is the primary regulator of adrenal glucocorticoid synthesis and secretion and only has affinity for MC2R. As the central actor in this hypothalamic-pituitary-adrenal (HPA) axis, ACTH is secreted in a circadian rhythm by the pituitary, and in pulses in response to stressful stimuli. ACTH acts at the adrenal gland to stimulate the synthesis and secretion of cortisol, which by negative feedback at the hypothalamic-pituitary axis regulates ACTH secretion. In ACTH-dependent Cushing's syndrome, pituitary and extra-pituitary tumors secrete excess ACTH, unregulated by negative feedback, which stimulates excessive cortisol secretion. Modulation of MC2R is attractive for the treatment of ACTH-dependent Cushing's syndrome.

### SUMMARY

Disclosed herein are methods of treating adrenocorticotropic hormone (ACTH)-dependent Cushing's syndrome.

In one aspect, disclosed herein is method of treating adrenocorticotropic hormone (ACTH)-dependent Cushing's syndrome in a human comprising orally administering once daily about 80 mg to about 160 mg of a compound having the structure of Compound 1, or a pharmaceutically acceptable salt thereof:

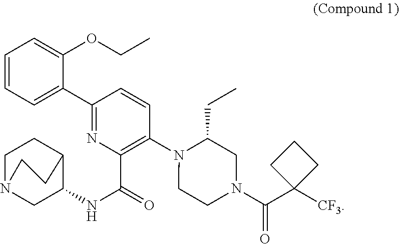 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

In some embodiments, the human with ACTH-dependent Cushing's syndrome comprises a pituitary adenoma.

In some embodiments, the human with ACTH-dependent Cushing's syndrome comprises an ectopic ACTH-secreting tumor. In some embodiments, the ectopic ACTH-secreting tumor is in the lungs, pancreas, thyroid, thymus gland, intestines, adrenal gland, paraganglia, or combinations thereof.

In some embodiments, treating ACTH-dependent Cushing's syndrome comprises lowering serum cortisol levels, lowering urine cortisol levels, or both. In some embodiments, treating ACTH-dependent Cushing's syndrome comprises lowering cortisol levels in the human by at least 10% from baseline.

In some embodiments, treating ACTH-dependent Cushing's syndrome comprises lowering levels of serum cortisol, androstenedione, or combinations thereof. In some embodiments, treating ACTH-dependent Cushing's syndrome comprises lowering levels of serum cortisol, androstenedione, or combinations thereof, by at least 10 from baseline.

In some embodiments, treating ACTH-dependent Cushing's syndrome comprises reducing levels of cortisol, androstenedione (A4), 17-hydroxyprogesterone (17-OHP), aldosterone, dehydroepiandrosterone sulfate (DHEAS), dehydroepiandrosterone (DHEA), or combinations thereof. In some embodiments, treating ACTH-dependent Cushing's syndrome comprises reducing levels of cortisol, androstenedione (A4), 17-hydroxyprogesterone (17-OHP), aldosterone, dehydroepiandrosterone sulfate (DHEAS), dehydroepiandrosterone (DHEA), or combinations thereof, by at least 10% from baseline.

In some embodiments, treating ACTH-dependent Cushing's syndrome comprises delaying or eliminating the need for removal of one or both adrenal glands.

In some embodiments, about 80 mg, about 120 mg, or about 160 mg of Compound 1, or a pharmaceutically acceptable salt thereof, is administered. In some embodiments, about 80 mg of Compound 1, or a pharmaceutically acceptable salt thereof, is administered. In some embodiments, about 120 mg of Compound 1, or a pharmaceutically acceptable salt thereof, is administered. In some embodiments, about 160 mg of Compound 1, or a pharmaceutically acceptable salt thereof, is administered. In some embodiments, Compound 1, or a pharmaceutically acceptable salt thereof, is administered once daily in the evening.

Articles of manufacture, which include packaging material, Compound 1, or a pharmaceutically acceptable salt thereof, within the packaging material, and a label that indicates that Compound 1, or a pharmaceutically acceptable salt thereof, is used for modulating the activity of a melanocortin receptor (e.g. melanocortin subtype-2 receptor (MC2R)), or for the treatment, prevention or amelioration of one or more symptoms of a disease or condition that would benefit from modulation of the activity of a melanocortin receptor (e.g. melanocortin subtype-2 receptor (MC2R)), such as ACTH-dependent Cushing's Syndrome, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION

Adrenocorticotropic hormone (ACTH) is a 39 amino acid peptide synthesized by anterior pituitary corticotropic cells by proteolytic cleavage of the proopiomelanocortin hormone (POMC). ACTH is the primary regulator of adrenal glucocorticoid (GC; cortisol in humans and most other species; corticosterone in rodents) synthesis and secretion. As the central actor in this hypothalamic-pituitary-adrenal (HPA) axis, ACTH is secreted by the pituitary in response to stressful stimuli and acts at the adrenal gland to stimulate the synthesis and secretion of cortisol. This stimulation is mediated through a highly specific G protein-coupled receptor (GPCR) which is expressed almost uniquely in the adrenal cortex. The receptor is the melanocortin 2 receptor (MC2R), and, along with ACTH, is part of the larger melanocortin system.

The melanocortin system comprises a family of five GPCRs (MC1R, MC2R, MC3R, MC4R, and MC5R); their natural agonists, the melanocortin peptides α-, β-, and γ-melanocyte-stimulating hormone (α-MSH, β-MSH, and γ-MSH) and ACTH; and endogenous melanocortin antagonists agouti and agouti-related protein (AGRP). The melanocortin receptors (MCRs) have different selectivities for endogenous agonist and antagonist peptides and are expressed in diverse tissues where they serve varied and discreet physiological functions (Gantz, I. and T. M. Fong, Am. J. Physiol. Endocrinol. Metab., 284: E468-E474, 2003).

It is possible to selectively modulate any one of the MCRs, or combinations thereof. In some embodiments, selectively modulating any one of the MCRs relative to the other MCRs, or combinations thereof, is useful in a variety of clinical applications. In some embodiments, selectively modulating any one of the MCRs relative to the other MCRs, or combinations thereof, reduces unwanted side effects in a variety of clinical applications. In one aspect, compounds described herein are antagonists of MC2R. In some embodiments, compounds described herein are selective antagonists for MC2R relative to other MCRs.

MC2R is a highly selective receptor for ACTH. Although ACTH can activate all five MCRs, at physiological levels, the sensitivity of the other receptors is not high enough to be activated, and ACTH selectively activates MC2R. Importantly, the other naturally occurring agonists α-MSH, β-MSH, and γ-MSH have no affinity for MC2R (Gantz, I. and T. M. Fong, Am. J. Physiol. Endocrinol. Metab., 284: E468-E474, 2003). The major function of MC2R is to stimulate the fasciculata cells of the adrenal cortex to synthesize and secret cortisol. MC2R requires the GPCR accessory protein MRAP (melanocortin 2 receptor protein) to be successfully secreted to the cell surface and as well as to function. MRAP is a small protein with a single transmembrane domain that forms an antiparallel homodimer in stable complex with MC2R and is necessary for both cell surface expression of MC2R and its ability to bind ACTH. MRAP can bind to any of the MCRs and affect their activities, but is only essential for MC2R activity. Binding of ACTH to the MC2R/MRAP complex on adrenal cortical cells activates Gₛ to elevate intracellular cAMP levels which in turn stimulates cortisol synthesis and secretion by regulating multiple steps in the steroidogenic pathway.

### Cushing's Disease

Cushing's syndrome (CS) is a constellation of clinical findings caused by prolonged exposure to excessive glucocorticoids. The clinical presentation is quite variable and may include muscle weakness, fatigue, easy bruising, weight gain, menstrual irregularities and striae. Facial rounding, plethora, and prominent fat deposits in the dorsal and supraclavicular areas are common. CS is associated with multiple cardiovascular, metabolic, skeletal, psychiatric, and dermatologic co-morbidities that greatly diminish quality of life and functional status. Mortality is increased due to cardiovascular and infectious complications. These associated comorbidities and mortality are decreased by early effective treatment.

CS may be caused by excessive amounts of adrenocorticotropic hormone (ACTH), either from a pituitary tumor (termed Cushing's disease, CD) or from a non-pituitary (i.e., ectopic) ACTH-secreting tumor (EAS). In these cases, excessive cortisol production is driven by normal or high ACTH levels that are not suppressed by cortisol negative feedback. Alternatively, CS may be caused by exogenous glucocorticoids or autonomous cortisol-secreting adrenal tumor(s). In these cases, the negative feedback effect of glucocorticoids on the normal pituitary corticotropes leads to a suppressed ACTH value. This difference in ACTH levels is used to distinguish between these two etiologic mechanisms.

Surgical resection of the causal lesion(s) is the optimal treatment of CS, with the potential for restoration of normal hypothalamic-pituitary-adrenal function. However, some patients cannot undergo surgery because of its risk, or if the tumor is occult. For others, such as those with metastatic disease or invasive pituitary lesions, surgery will not be curative. Also, while transsphenoidal surgery leads to an initial normalization of cortisol levels in 70% to 90% of patients in the hands of expert neurosurgeons, permanent cure rates are estimated to be 60% to 70%. Recurrences of CD have been reported months to decades following surgical results initially thought to be curative. Primary pharmacologic treatment is needed for all of these patients, and adjunctive medical treatment is needed for CD patients undergoing radiation, until the tumor is controlled.

Current medical treatment of any cause of CS may be directed to the adrenal gland via inhibition of enzymes leading to cortisol synthesis (osilodrostat, ketoconazole, levoketoconazole, metyrapone, and etomidate) or to the glucocorticoid receptor (to antagonize glucocorticoid action: mifepristone). Therapy of CD also includes agents that inhibit ACTH secretion: cabergoline and pasireotide. These agents are associated with toxicities that may preclude long-term use at effective doses (e.g., hypogonadism in men, or liver function abnormalities in patients taking ketoconazole), and the need for multiple agents to treat severe hypercortisolism increases the risk of adverse events.

In healthy people and those with ACTH-dependent CS, ACTH acts at the adrenal gland to stimulate the synthesis and secretion of cortisol through activation of MC2R. Cortisol is the body's main stress hormone and excess amounts can cause significant increases in mortality and morbidity. Corticotroph adenomas are small, usually slow growing, benign tumors that normally come to clinical attention as a result of the effects of glucocorticoid excess, rather than because of the physical effects of an expanding tumor. CD presents much more commonly in women, and usually between 30 and 50 years of age. CD often takes many years to diagnose with an estimated diagnosis time of 38 months, and may well be under-diagnosed in the general population as many of its symptoms such as lethargy, depression, obesity, hypertension, hirsutism and menstrual irregularity can be incorrectly attributed to other more common disorders.

First-line therapy for ACTH-dependent CS is surgical and involves removal of either the ACTH-secreting tumor in the pituitary or, in severe cases, the adrenal glands themselves. As surgery is often unsuccessful, contraindicated, or delayed, pharmacological therapy for these patients becomes necessary. Adrenal enzyme inhibitors (e.g., metyrapone and ketoconazole) prevent the synthesis of cortisol and can improve symptoms but suffer from mechanistic side effects. For example, metyrapone is associated with hirsutism in women and patients must be monitored carefully to avoid hypoadrenalism, hypokalemia, and hypertension. Ketoconazole often requires progressively increasing dosage to maintain disease control, but this is ultimately limited by the hepatotoxicity of the drug. In addition, it is a potent inhibitor of one of the most important drug metabolizing enzymes in the liver, CYP3A4, resulting in the potential for negative drug-interactions as a side effect. Mifepristone, a potent glucocorticoid receptor antagonist, is approved for control of hyperglycemia in Cushing's syndrome, but is difficult to titrate and has significant liabilities due to its potent anti-progesterone activity.

In some embodiments, Compound 1, or a pharmaceutically acceptable salt thereof, is used in the treatment of Cushing's syndrome. In some embodiments, Compound 1, or a pharmaceutically acceptable salt thereof, is used in the treatment of ACTH-dependent Cushing's syndrome. In some embodiments, Compound 1, or a pharmaceutically acceptable salt thereof, is used in the treatment of Cushing's disease.

### Compound 1

Compound 1 refers to 6-(2-ethoxyphenyl)-3-((R)-2-ethyl-4-(1-(trifluoromethyl)cyclobutane-1-carbonyl) piperazin-1-yl)-N—((S)-quinuclidin-3-yl) picolinamide, which has the chemical structure shown below.

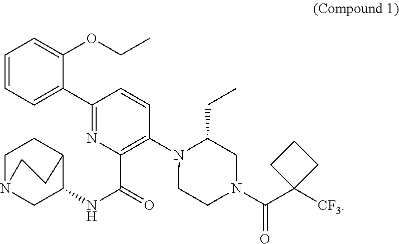 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Compound 1 is also referred to as N-[(3S)-1-azabicyclo[2.2.2]octan-3-yl]-6-(2-ethoxyphenyl)-3-[(2R)-2-ethyl-4-[1-(trifluoromethyl)cyclobutanecarbonyl]piperazin-1-yl]pyridine-2-carboxamide.

### Preclinical Studies

Compound 1 is a potent oral, non-peptide, small molecule MC2R antagonist (Kᵢ<10 nM) that is >2900-fold selective over other human melanocortin receptor subtypes. Compound 1 potently blocked ACTH binding to the receptor and ACTH-mediated G-protein activation and subsequent cAMP production. In a screen for potential off-target activities it had weak antagonist and no agonist activity against the 5-HT_{1B} receptor with more than 2200-fold selectivity for hMC2R over 5-HT_{1B}.

In vitro studies evaluated the metabolism of Compound 1 in plasma, liver microsomes, and/or 9000 g supernatant fraction of the liver homogenate (liver S9) from various species (including human). The primary metabolic pathways known to be involved in metabolic clearance of Compound 1 are oxidative metabolism, resulting in formation of hydroxylated and desmethyl metabolites. CYP3A4 plays a key role in catalyzing these reactions; CYP2D6 and CYP2C8 may also have a minor contribution. CYP1A2, CYP2B6, CYP2C8, CYP2C9, CYP2C19 were not involved.

Oral administration of Compound 1 dose-dependently suppressed corticosterone (murine equivalent to cortisol in human) secretion after 1 μg/kg ACTH (1-24) at doses of 3 and 10 mg/kg in male and female rats. After ACTH administration at 1, 4, 24, and 48 hours after a single dose of Compound 1 there was a dose-dependent suppression of corticosterone at 1 and 4 hours only.

Disease models mimicking states of ACTH excess and hypercortisolism included rats administered exogenous ACTH (1-24) via subcutaneous osmotic pump over 7 days and mice implanted with ACTH-secreting corticotroph AtT-20 tumor cells; animals received daily oral doses of placebo or Compound 1. In each model, corticosterone values on Days 1 and 7 showed a significant dose-dependent decrease to a maximum of 92%, with dose-dependent adrenal gland atrophy. Compound 1 also reduced ACTH-stimulated aldosterone levels but did not affect Angiotensin-II stimulation of aldosterone, which accounts for most aldosterone production. However, six days after discontinuation of the highest dose of Compound 1 (75 mg/kg for 15 days), Ang-II stimulated aldosterone was suppressed, but sodium and potassium levels were normal.

The overall nonclinical safety of Compound 1 was assessed in 28-day and 3-month repeat dose general toxicity studies in rat and dog and in a battery of in vitro and in vivo genotoxicity and safety pharmacology studies. The repeat-dose toxicity studies showed effects in the rat lungs and dog adrenal gland and liver, which were further evaluated. Target organs of toxicity identified in repeat-dose toxicity studies included (1) foamy macrophages in the lungs in the 28-day rat study and (2) adrenal and liver atrophy in the 3-month dog study. Findings in the rat lung were confirmed not to be consistent with drug-induced PLD. The adrenal gland and liver findings in the dog were shown to be mitigated with hydrocortisone supplementation, demonstrating that these findings were an extension of pharmacology. No findings indicating significant genotoxic risk or adverse effects on core organ systems, including cardiovascular, central nervous, and respiratory systems, were observed.

### Methods of Treatment

In certain aspects, disclosed herein are methods of treating ACTH-dependent Cushing's syndrome (CS) in a subject. ACTH-dependent Cushing's syndrome comprises Cushing's disease (CD) and ectopic ACTH syndrome (EAS). In some embodiments, the treatment comprises reducing cortisol levels. In some embodiments, the treatment comprises reducing cortisol levels driven by endogenous ACTH. In some embodiments, the subject is a human.

In some embodiments, treating ACTH-dependent Cushing's syndrome comprises reducing the level of cortisol in the human. In some embodiments, the methods comprise reducing levels of endogenous cortisol. In some embodiments, the levels of endogenous cortisol are reduced in the blood, serum, saliva, or urine. In some embodiments, the methods comprise reducing cortisol. In some embodiments, the levels of cortisol are reduced in the blood, serum, saliva, or urine. In some embodiments, blood cortisol levels are reduced. In some embodiments, serum cortisol levels are reduced. In some embodiments, treating ACTH-dependent Cushing's syndrome comprises lowering serum cortisol levels, lowering urine cortisol levels, or both. In some embodiments, salivary levels of cortisol are reduced. In some embodiments, urine free cortisol (UFC) levels are reduced. In some embodiments, cortisol secretory capacity is reduced as determined by an ACTH-stimulation test. In some embodiments, treating ACTH-dependent Cushing's syndrome comprises lowering cortisol levels in the human by at least 10%, at least 15%, at least 20%, at least 25%, at least 30%, or more than 30% from baseline. In some embodiments, treating ACTH-dependent Cushing's syndrome comprises lowering cortisol levels in blood, saliva, or urine by at least 10%, at least 15%, at least 20%, at least 25%, at least 30%, or more than 30% from baseline. In some embodiments, treating ACTH-dependent Cushing's syndrome comprises lowering cortisol levels in blood, urine, or both by at least 10%, at least 15%, at least 20%, at least 25%, at least 30%, or more than 30% from baseline. In some embodiments, treating ACTH-dependent Cushing's syndrome comprises lowering cortisol levels in blood by at least 10%, at least 15%, at least 20%, at least 25%, at least 30%, or more than 30% from baseline. In some embodiments, treating ACTH-dependent Cushing's syndrome comprises lowering cortisol levels in serum by at least 10%, at least 15%, at least 20%, at least 25%, at least 30%, or more than 30% from baseline. In some embodiments, treating ACTH-dependent Cushing's syndrome comprises lowering cortisol levels in saliva by at least 10%, at least 15%, at least 20%, at least 25%, at least 30%, or more than 30% from baseline. In some embodiments, treating ACTH-dependent Cushing's syndrome comprises lowering cortisol levels in urine by at least 10%, at least 15%, at least 20%, at least 25%, at least 30%, or more than 30% from baseline. In some embodiments, the levels of cortisol are reduced compared to the levels of cortisol before treatment. In some embodiments, the levels of cortisol are reduced to that of a human without one of the diseases described herein. That is, in some embodiments, the levels of cortisol are restored to a normal level in the human.

In some embodiments, treating ACTH-dependent Cushing's syndrome comprises reducing levels of at least one of the following in the subject: serum cortisol, androstenedione, or combinations thereof. In some embodiments, treating ACTH-dependent Cushing's syndrome comprises lowering levels of serum cortisol, androstenedione, or combinations thereof. In some embodiments, levels of serum cortisol, or androstenedione are reduced by at least 10%, at least 15%, at least 20%, at least 25%, at least 30%, or more than 30% from baseline. In some embodiments, treating ACTH-dependent Cushing's syndrome comprises lowering levels of serum cortisol, androstenedione, or combinations thereof, by at least 10%, at least 15%, at least 20%, at least 25%, at least 30%, or more than 30% from baseline.

In some embodiments, treating ACTH-dependent Cushing's syndrome comprises reducing levels of cortisol, androstenedione (A4), 17-hydroxyprogesterone (17-OHP), aldosterone, dehydroepiandrosterone sulfate (DHEAS), dehydroepiandrosterone (DHEA), or combinations thereof. In some embodiments, the levels of any one of cortisol, androstenedione (A4), 17-hydroxyprogesterone (17-OHP), aldosterone, dehydroepiandrosterone sulfate (DHEAS), or dehydroepiandrosterone (DHEA) is reduced by at least 10%, at least 15%, at least 20%, at least 25%, at least 30%, or more than 30% from baseline. In some embodiments, treating ACTH-dependent Cushing's syndrome comprises reducing levels of cortisol, androstenedione (A4), 17-hydroxyprogesterone (17-OHP), aldosterone, dehydroepiandrosterone sulfate (DHEAS), dehydroepiandrosterone (DHEA), or combinations thereof, by at least 10%, at least 15%, at least 20%, at least 25%, at least 30%, or more than 30% from baseline.

In some embodiments, treating ACTH-dependent Cushing's syndrome comprises treating one or more symptoms of Cushing's syndrome. In some embodiments, treating ACTH-dependent Cushing's syndrome comprises reducing growth of fat pads (collarbone, back of neck, face and trunk), excessive sweating, dilation of capillaries, thinning of the skin, muscle weakness, hirsutism, depression/anxiety, hypertension, osteoporosis, insulin resistance, hyperglycemia, heart disease, lethargy, obesity (including truncal, abdominal, or central obesity), menstrual irregularity, or combinations thereof. In some embodiments, treating ACTH-dependent Cushing's syndrome comprises reducing growth of fat pads (collarbone, back of neck, and face). In some embodiments, treating ACTH-dependent Cushing's syndrome comprises reducing excessive sweating. In some embodiments, treating ACTH-dependent Cushing's syndrome comprises reducing dilation of capillaries. In some embodiments, treating ACTH-dependent Cushing's syndrome comprises reducing thinning of the skin. In some embodiments, treating ACTH-dependent Cushing's syndrome comprises reducing muscle weakness. In some embodiments, treating ACTH-dependent Cushing's syndrome comprises reducing hirsutism. In some embodiments, treating ACTH-dependent Cushing's syndrome comprises reducing depression/anxiety. In some embodiments, treating ACTH-dependent Cushing's syndrome comprises reducing hypertension. In some embodiments, treating ACTH-dependent Cushing's syndrome comprises reducing osteopenia, osteoporosis, or fracture risk. In some embodiments, treating ACTH-dependent Cushing's syndrome comprises reducing insulin resistance, risk of hyperglycemia, diabetes mellitus, or the need for glucose-lowering medications. In some embodiments, treating ACTH-dependent Cushing's syndrome comprises reducing hypertension or the need for blood pressure lowering medicines.

In some embodiments, treating EAS comprises reducing blood potassium abnormalities or the risk for thrombosis.

In some embodiments, treating ACTH-dependent Cushing's syndrome comprises reducing hyperglycemia. In some embodiments, treating ACTH-dependent Cushing's syndrome comprises reducing heart disease. In some embodiments, treating ACTH-dependent Cushing's syndrome comprises reducing lethargy. In some embodiments, treating ACTH-dependent Cushing's syndrome comprises reducing obesity. In some embodiments, treating ACTH-dependent Cushing's syndrome comprises reducing menstrual irregularity, infertility, hirsutism, acne, or androgen-drive hair loss in women.

In some embodiments, treating ACTH-dependent Cushing's syndrome comprises delaying or eliminating the need for removal of one or both adrenal glands.

### Subjects

In some embodiments, the subject is a human. In some embodiments, the subject has a disease associated with excess ACTH. In some embodiments, the subject has ACTH-dependent Cushing's syndrome.

In some embodiments, the human with ACTH-dependent Cushing's syndrome has at least two of the following: (i) failure of serum cortisol to suppress to <1.8 μg/dL during a 1 mg Dexamethasone Suppression Test (DST); (ii) at least 4 consecutive bedtime salivary cortisol values>ULN, wherein each value was obtained at an interval of about 7 to about 14 days; and (iii) at least 4 consecutive 24-hour urine free cortisol (UFC) values>1.3×ULN without any lower values, wherein each value was obtained at an interval of about 7 to about 14 days.

In some embodiments, the human has elevated serum cortisol in the low dose dexamethasone suppression test. In some embodiments, the human has serum cortisol level of 1.8 mcg/dL or higher the morning following night-time administration of 1 mg of dexamethasone.

In some embodiments, the human has late night salivary cortisol level higher than the ULN for a human. In some embodiments, the human has late night salivary cortisol level at least 1.5-fold, 2.0-fold, 2.5-fold or more of ULN. In some embodiments, the ULN for late night salivary cortisol level is about 100 ng/dL.

In some embodiments, the human has 24-hour urine free cortisol (UFC) level higher than the ULN for a human. In some embodiments, the human has 24-hour UFC at least 1.3-fold of ULN. In some embodiments, the human has 24-hour UFC at least 1.5-fold, 2.0-fold, 2.5-fold or more of ULN. In some embodiments, the human has 24-hour UFC higher than about 50 mcg/day, 100, mcg/day, 150 mcg/day, 200 mcg/day or more in a 24-hour urinary cortisol test. In some embodiments, the ULN for 24-hour urinary cortisol excretion is about 50-55 mcg/day.

In some embodiments, ACTH-dependent Cushing's syndrome comprises: (a) late-night serum cortisol>7.5 μg/dL, or bedtime salivary cortisol>ULN, wherein the cortisol levels are measured prior to the initiation of treatment with Compound 1, or a pharmaceutically acceptable salt thereof; and (b) early morning plasma ACTH>10 pg/mL, wherein the plasma ACTH is measured prior to the initiation of treatment with Compound 1, or a pharmaceutically acceptable salt thereof.

In some embodiments, the human with ACTH-dependent Cushing's syndrome has a pituitary gland tumor (pituitary adenoma) or corticotroph adenoma. In some embodiments, the human with ACTH-dependent Cushing's syndrome has a pituitary gland tumor (pituitary adenoma). In some embodiments, the human with ACTH-dependent Cushing's syndrome has a corticotroph adenoma. In some embodiments, the pituitary adenoma or corticotroph adenoma is benign. In some embodiments, treating ACTH-dependent Cushing's syndrome comprises corticotroph adenoma.

In other embodiments, the human has an ectopic ACTH-secreting tumor. In some embodiments, the human has an ACTH-secreting tumor in an organ that does not normally produce ACTH. In some embodiments, the ACTH-secreting tumor is in the lungs, pancreas, thyroid, thymus gland, intestines, adrenal gland, paraganglia, or a combination thereof. In some embodiments, the ACTH-secreting tumor is cancerous. In some embodiments, the ACTH-secreting tumor is benign.

### Dosage and Administration

In one embodiment, Compound 1, or a pharmaceutically acceptable salt thereof, are used in the preparation of medicaments for the treatment of ACTH-dependent Cushing's syndrome. Methods for the treatment of ACTH-dependent Cushing's syndrome in a subject in need of such treatment, involve administration of pharmaceutical compositions that include at least Compound 1 or a pharmaceutically acceptable salt thereof, in therapeutically effective amounts to said subject.

In some embodiments, Compound 1, or a pharmaceutically acceptable salt thereof, is administered orally to the human. In some embodiments, Compound 1, or a pharmaceutically acceptable salt thereof, is administered to the human on a continuous dosing schedule. In some embodiments, Compound 1, or a pharmaceutically acceptable salt thereof, is administered to the human on a continuous daily dosing schedule.

The term “continuous dosing schedule” refers to the administration at regular intervals. In some embodiments, continuous dosing schedule refers to the administration at regular intervals without any drug holidays from the particular therapeutic agent.

The term “continuous daily dosing schedule” refers to the administration of a particular therapeutic agent every day at roughly the same time each day. In some embodiments, daily administration is once a day.

In some embodiments, Compound 1, or a pharmaceutically acceptable salt thereof, is administered once-a-day. In some embodiments, Compound 1, or a pharmaceutically acceptable salt thereof, is administered once-a-day in the evening.

In some embodiments, Compound 1, or a pharmaceutically acceptable salt thereof, is administered in an amount equivalent to about 50 mg to about 200 mg of Compound 1. In some embodiments, Compound 1, or a pharmaceutically acceptable salt thereof, is administered in an amount equivalent to about 80 mg to about 160 mg of Compound 1.

In some embodiments, Compound 1, or a pharmaceutically acceptable salt thereof, is administered in amount equivalent to about: 80 mg, 120 mg, or 160 mg of Compound 1. In some embodiments, Compound 1, or a pharmaceutically acceptable salt thereof, is administered in amount equivalent to about 80 mg of Compound 1. In some embodiments, Compound 1, or a pharmaceutically acceptable salt thereof, is administered in amount equivalent to about 120 mg of Compound 1. In some embodiments, Compound 1, or a pharmaceutically acceptable salt thereof, is administered in amount equivalent to about 160 mg of Compound 1.

In certain instances, it is appropriate to administer at least one additional therapeutic agent in combination with Compound 1, or a pharmaceutically acceptable salt thereof.

### Adrenal Glucocorticoid “Block and Replace” Therapy

In one specific embodiment, Compound 1, or a pharmaceutically acceptable salt thereof, is co-administered with one or more additional therapeutic agents, wherein the one or more additional therapeutic agents are selected from a glucocorticoid, a mineralocorticoid, or combination thereof. In some embodiments, administration of Compound 1, or a pharmaceutically acceptable salt thereof, comprises reducing exogenous glucocorticoid dose requirements for the human. Example glucocorticoids contemplated for co-administration include, but are not limited to, beclomethasone, betamethasone, budesonide, cortisone, dexamethasone, ethamethasoneb, hydrocortisone, methylprednisolone, prednisolone, prednisone, triamcinolone, or the like. Example mineralocorticoids contemplated for co-administration include, but are not limited to, fludrocortisone and the like.

In some embodiments of the treatment of ACTH-dependent Cushing's syndrome, Compound 1, or a pharmaceutically acceptable salt thereof, is co-administered with a second therapeutic agent, wherein the second therapeutic agent is a glucocorticoid (i.e. “block and replace” therapy). In some embodiments, the glucocorticoid is beclomethasone, betamethasone, budesonide, cortisone, dexamethasone, ethamethasoneb, hydrocortisone, methylprednisolone, prednisolone, prednisone, or triamcinolone. In some embodiments of the treatment of ACTH-dependent Cushing's syndrome, Compound 1, or a pharmaceutically acceptable salt thereof, is co-administered with a low-dose glucocorticoid. In some embodiments, low-dose glucocorticoid comprises less than or equal to 20 mg/day cortisone equivalent. In some embodiments, low-dose glucocorticoid comprises less than or equal to 15 mg/day cortisone equivalent. In some embodiments, low-dose glucocorticoid comprises less than or equal to 10 mg/day cortisone equivalent. In some embodiments, low-dose glucocorticoid comprises less than or equal to 5 mg/day cortisone equivalent.

In some embodiments of the treatment of ACTH-dependent Cushing's syndrome, the method further comprises the administration of adrenal glucocorticoid replacement therapy if serum cortisol is <5 μg/dL; wherein the serum cortisol is measured in early morning. In some embodiments, early morning is between about 0530 and about 0800. In some embodiments, adrenal glucocorticoid replacement therapy comprises hydrocortisone. In some embodiments, hydrocortisone is administered at a physiologic hydrocortisone replacement dosage. In some embodiments, hydrocortisone is administered at a supraphysiologic hydrocortisone replacement dosage.

In some embodiments of the treatment of ACTH-dependent Cushing's syndrome, the method further comprises the administration of mineralocorticoid replacement therapy. In some embodiments, mineralocorticoid replacement therapy comprises fludrocortisone.

### Pharmaceutical Compositions

In some embodiments, Compound 1, or a pharmaceutically acceptable salt thereof, is formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, pharmaceutical compositions suitable for oral administration are presented as discrete units such as capsules or tablets each containing a predetermined amount of Compound 1, or a pharmaceutically acceptable salt thereof; as a solution or a suspension in an aqueous liquid or a non-aqueous liquid; or as an oil-in-water liquid emulsion or a water-in-oil liquid emulsion.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The term “pharmaceutical combination” as used herein, means a product that results from the mixing or combining of more than one active ingredient and includes both fixed and non-fixed combinations of the active ingredients. The term “fixed combination” means that the active ingredients, e.g., Compound 1, or a pharmaceutically acceptable salt thereof, and a co-agent, are both administered to a patient simultaneously in the form of a single entity or dosage. The term “non-fixed combination” means that the active ingredients, e.g., Compound 1, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The section headings used herein are for organizational purposes only and are not to be construed as limiting the subject matter described.

### EXAMPLES

The following examples are included for illustrative purposes only and are not intended to limit the scope of the invention.

### Example 1: a Phase 1b/2a Open-Label Multiple-Ascending Dose Exploratory Study of Compound 1 in ACTH Dependent Cushing's Syndrome

A non-limiting example of a clinical trial of an ACTH antagonist in humans is described below.

### Study Description

This is a Phase 1b/2a, first-in-disease, open-label, uncontrolled, multiple-ascending dose exploratory study to evaluate safety, tolerability, and pharmacokinetics (PK) of Compound 1 in participants with ACTH-dependent Cushing's syndrome (Cushing's disease or ectopic ACTH syndrome).

The study will require a 14-day hospitalization during which participants will receive oral Compound 1 once daily for 10 days, followed by monitoring during 4 ‘wash-out’ days. Vital signs and safety laboratories will be obtained.

### Objectives

Primary Objectives: To evaluate the safety and tolerability of Compound 1 in participants with ACTH-dependent Cushing's syndrome. To evaluate the pharmacokinetics of Compound 1 in participants with ACTH-dependent Cushing's syndrome.

Secondary Objective: To document change in early morning (~0530-0800) serum cortisol.

Exploratory Objectives: To demonstrate lowering of urinary free cortisol (UFC) in participants with ACTH-dependent Cushing's syndrome. To evaluate the effect of Compound 1 on ACTH, other adrenal steroids, clinical features, and biochemistry.

### Endpoints

Primary Endpoint: Assessment of safety and tolerability of Compound 1 in participants with ACTH-dependent Cushing's syndrome:
Daily assessment for adrenal insufficiency with early morning serum cortisol, adrenal insufficiency questionnaire, in person visit
Measurement of CBC with differential, electrolytes, BUN, glucose, creatinine, liver function tests
Pre- and posttreatment measurement of insulin and pituitary function tests
Daily ECG assessments
Aldosterone and renin activity

### Pharmacokinetic Endpoints:

Maximum plasma concentration on Day 1 and Day 10 (Cₘₐₓ).
Time to Cₘₐₓ on Day 1 and Day 10 (tₘₐₓ)
Area under the plasma concentration-time curve on Day 1 and Day 10 (AUC₀₋₂₄)
Accumulation index for Cₘₐₓ
Accumulation index for AUC
Terminal half-life

Secondary Endpoint: Change from Baseline in early morning (~0530-0800) serum cortisol on Day 11.

Exploratory Endpoints: Except where otherwise noted, all exploratory endpoints assess change from Baseline to Day 11. Change in UFC. 30% decrease in UFC. Change in morning (~0530-0800) plasma ACTH. Change in 24-hour area under the plasma concentration-time curve (AUC) in serum cortisol, 11-oxygenated androgens (11-ketotestosterone [11KT] and 11β-hydroxyandrostenedione [11OHΔ4A]), and plasma ACTH. Percentage of participants who normalize (≤upper limit of normal [ULN]) UFC on Day 11. Day 10-11 UFC as percentage of ULN. Correlation between baseline pituitary lesion size and UFC response.

Change from Baseline morning values only to Day 11 morning values and % of each cohort with normal values at Baseline and Day 11 for the following:
Serum and urinary potassium (mEq/L)
Plasma glucose (mg/dL) and fructosamine (μmol/L)
Insulin sensitivity (Homeostatic Model Assessment of Insulin Resistance [HOMA-IR])
Systolic blood pressure (BP) (mm Hg)
Diastolic BP (mm Hg)
Weight (kg) and waist circumference (cm)

### Study Population

Up to 40 participants, 18-75 years at screening with a confirmed diagnosis of active ACTH-dependent Cushing's syndrome (Cushing's disease or ectopic ACTH syndrome), to achieve 6 completers for each dose cohort.

### Inclusion Criteria:

To participate in this study, participants must have active ACTH-dependent Cushing's syndrome. They may be participants currently under the care of the research team who are awaiting surgery or radiation therapy, or who have failed surgery and/or are awaiting a response to radiotherapy. Participants who do not meet remission criteria within five days after resection of a possible ACTH-producing tumor may not initiate screening until 6 weeks after the surgical date. Alternatively, they may be participants who will be admitted for evaluation of the cause of Cushing's syndrome.

To be eligible to participate in this study, an individual must meet all of the following criteria: provision of signed and dated informed consent form; ability to understand and willingness to sign a written informed consent document; willingness to comply with all study procedures and interventions; ability to take oral medication; male or female, aged 18-75 years; vaccination against COVID-19 according to current CDC recommendations; evidence in support of the diagnosis of Cushing's syndrome, defined as meeting 2 of the following 3 criteria performed up to 56 days prior to Day 1: (i) Failure of serum cortisol to suppress to ≤1.8 μg/dL during a 1 mg Dexamethasone Suppression Test (DST) may be used in all participants except for women taking oral estrogen-containing medication, (ii) at least 4 (at 7-14-day intervals) consecutive bedtime salivary cortisol values >ULN, (iii) at least 4 (at 7-14-day intervals) consecutive 24-hour urine free cortisol (UFC) values >1.3×ULN without any lower values (however, for participants with previously confirmed ACTH-dependent Cushing's syndrome who are awaiting surgery or radiation therapy, or who have failed surgery or are awaiting the effects of radiation therapy, a DST is not required and only one additional elevated UFC or bedtime salivary cortisol value is required within three weeks of Day 1); evidence of ‘active’ ACTH-dependent Cushing's syndrome within 14 days of Day 1, defined as: (i) Late-night (approximately 2300-0000) serum cortisol >7.5 μg/dL, or bedtime salivary cortisol >ULN, and (ii) early morning (~0530-0800) plasma ACTH>10 pg/mL. Participants with documented ACTH-dependent Cushing's syndrome taking short-acting steroidogenesis inhibitors (ketoconazole, levoketoconazole, osilodrostat, cabergoline or metyrapone) may participate after a 14-day washout period, if they meet other study inclusion criteria.

### Exclusion Criteria:

An individual who meets any of the following criteria will be excluded from participation in this study: women who are pregnant or lactating; history of bilateral adrenalectomy; if previously experienced adrenal insufficiency when taking Compound 1 on this study; previous pituitary MRI findings of a lesion within 3 mm of the optic chiasm; presence of any known malignancy; a UFC more than 5-fold ULN; use of mitotane; use of prohibited prescribed or non-prescribed medications and/or non-medications/alternative medicinal products within 7 days prior to Screening and is not willing to forego use of these substances during the study; use of medications that are strong inducers of cytochrome (CYP) 3A4 within 30 days prior to the day of enrollment; use of medications or ingestion of food that are strong or moderate inhibitors of CYP3A4 within 14 days prior to the day of enrollment; use of medications that are strong or moderate inducers of P-gp within 14 days prior to first dose of study drug; use of medications that are strong or moderate inhibitors of P-gp within 14 days prior to first dose of study drug; use of any investigational drug within the past 30 days or for 5 half-lives, whichever is longer, prior to the day of Compound 1 administration; any condition that in the opinion of the Investigator would jeopardize the participant's appropriate participation in this study; previous unsuccessful surgery for Cushing's syndrome within 6 weeks; and abnormal baseline EKG in any of the following ways: (a) based on the average of triplicate ECGs, QTcF >500 msec (or QTcF >530 msec in participants with a bundle branch block) repeated on a second set of ECGs at least 2 hours apart and confirmed by the Principal Investigator; (b) any ventricular tachyarrhythmia associated with symptoms of hemodynamic response; (c) sustained ventricular tachycardia (lasting >30 sec) irrespective of symptoms; (d) Torsades de pointes; (e) cardiac arrest; (f) pause >5 sec; (g) Type II second degree block or third-degree atrioventricular block; (h) clinically significant, symptomatic bradycardia; (i) any supraventricular tachyarrhythmia associated with symptoms of hemodynamic response.

### Lifestyle Considerations:

During this study, participants are asked to do the following: refrain from consumption of Seville oranges, grapefruit, pomelos, exotic citrus fruits, grapefruit hybrids, or more than 8 ounces of juices containing these fruits, starting 7 days before the first dose of Compound 1 until after the final dose; refrain from the consumption of red wine starting 2 days before the first dose of Compound 1 until after the final dose; participants who use tobacco products will be instructed that use of nicotine-containing products will not be permitted within the Clinical Center; abstain from strenuous exercise throughout the duration of the study.

### Overall Study Design

This is a single-site phase 1b/2a, first-in-disease, open-label, uncontrolled, multiple-ascending dose exploratory study to evaluate safety, tolerability, pharmacokinetics, and signals of efficacy of Compound 1 (an ACTH receptor antagonist) in participants with active ACTH-dependent Cushing's syndrome.

Six people will enter each dose cohort, as consecutively available. Participants may enter more than one dose cohort. The study requires a 14-day hospitalization during which participants will receive oral Compound 1 once daily for 10 days, followed by monitoring during 4 ‘wash-out’ days. Vital signs and alternate day safety laboratories will be obtained. Blood levels of cortisol, and 24-hour UFC will be the main measures of signals of efficacy. There will be one safety follow-up review between Days 24 and 31.

### Justification for Dose

The starting dose of 80 mg is based on available toxicology and clinical data from healthy volunteer studies. Lower doses are not likely to adequately control serum and urinary cortisol in participants with Cushing's syndrome in whom basal ACTH and cortisol levels will be significantly higher than those seen in healthy volunteers. The maximum proposed daily dose is 160 mg. Based on the population PK model, this dose is projected to have a mean steady state Cₘₐₓ and AUC₀₋₂₄ of 2510 ng/ml and 31,800 ng×h/mL, respectively. The histopathology findings (i.e., lung, adrenal gland, and liver) observed in the rat and dog repeat-dose toxicity studies typically occur only after repeat dose administration and are likely to correlate with total exposure (AUC) and not with peak exposure (Cₘₐₓ). Therefore, it is anticipated that Compound 1 can be safely administered to healthy participants with ACTH-dependent Cushing's syndrome at planned doses of up to 160 mg.

### Intervention

Compound 1 oral tablet once daily for 10 days. Three ascending dose cohorts are anticipated: 80, 120, and 160 mg per day. The first cohort will receive a dose of 80 mg Subsequent cohorts may receive 120 and 160 mg. Participant Duration: up to 45 days.

Sequential, open-label, 10-day fixed-dose cohorts of 6 participants:
Cohort 1: Compound 1 80 mg once daily
Cohort 2: Compound 1 120 mg once daily
Cohort 3: Compound 1 160 mg once daily

Participants will receive Compound 1 at the appropriate dose for their cohort as a single daily dose for 10 days at approximately 8 am. Tablets will be taken orally with 240 mL of water. The drug may be taken before or after breakfast, as there is minimal effect of food on absorption.

### Glucocorticoid Deficiency

Glucocorticoid deficiency is an adverse event of special interest (AESI) that may occur as a result of the pharmacologic action of the Compound 1. Glucocorticoid deficiency will be treated with glucocorticoid replacement at doses determined by the Investigator. Mild to moderate glucocorticoid deficiency will not result in modification of study drug as long as there is a prompt clinical response of any related symptoms or signs to glucocorticoid treatment in the opinion of the investigator.

Glucocorticoid deficiency was observed during treatment with Compound 1 in a previous study involving healthy volunteers, consistent with the pharmacologic action of the study drug. It has therefore been identified as an AESI in this study and may lead to dose limiting toxicity.

Mild to moderate glucocorticoid deficiency is defined as inappropriately low basal or ACTH stimulated serum cortisol concentrations with or without concomitant related symptoms or signs (e.g., nausea, anorexia, hyponatremia). In all participants with an early morning (~0530-0800) serum cortisol <5 μg/dL, it is recommended that HC therapy be initiated, regardless of symptoms.

Severe (hypotensive) glucocorticoid deficiency is defined as an acute deterioration in health status including, but not limited to, the above signs and symptoms associated with absolute hypotension (systolic blood pressure <100 mm Hg) or relative hypotension (systolic blood pressure ≥20 mm Hg lower than usual), with features that resolve within 1 to 2 hours after parenteral HC administration (i.e., a marked resolution of hypotension within 1 hour and improvement in clinical symptoms over a period of 2 hours).

Participants who are diagnosed with glucocorticoid deficiency will receive hydrocortisone (and potential other interventions) according to severity. The diagnosis and management of glucocorticoid insufficiency and supervision of hydrocortisone therapy will be overseen by the Investigator and participants will be treated per standard of care.

Prior to initiating HC therapy, if possible, blood should be collected for measurement of serum cortisol, ACTH, aldosterone, renin activity, chemistry (sodium, potassium, glucose), and any scheduled hormone measurements.

For asymptomatic participants or participants with mild symptoms, HC dosing should be administered at a physiologic HC replacement dosage.

Study drug dosing may continue, under careful supervision, if 1 of the following scenarios occur: (1) participant is on physiologic HC replacement and asymptomatic; OR (2) any symptoms or signs related to mild to moderate glucocorticoid deficiency responded promptly to glucocorticoid treatment as determined by the Investigator.

### Screening Procedures

Cushing's syndrome will be determined by the results from 1 mg dexamethasone suppression test, 4 or more elevated bedtime salivary cortisol levels and/or four or more elevated UFC>1.3-fold normal; OR by one elevated UFC or salivary cortisol value within 21 days, for patients whose Cushing's syndrome was confirmed before 56 days of enrollment.

Participants with active Cushing's syndrome will undergo the following Screening activities only after signing the consent for this study: pregnancy test, triplicate 12-lead ECG, and measurement of electrolytes, glucose, blood urea nitrogen, creatinine, alanine transaminase (ALT), aspartate transaminase (AST), gamma glutamyl transferase (GGT), complete blood count, late night serum cortisol, pituitary MRI, and morning plasma ACTH.

### Study Evaluations & Procedures

The study requires collection of biospecimens before, during and after 10 days administration of study drug Compound 1.

Physical examination will include measurement of height, weight, waist circumference, and vital signs (blood pressure, pulse, temperature, respiratory rate) using automated pulse and BP measurement with the correct size cuff, assessment of the thyroid gland, heart and lung sounds, abdominal sounds, tenderness, organ size, presence of abnormal adipose (dorsocervical, supraclavicular, temporal) depots or marked central adiposity, presence of acne, hirsutism, striae or hyperpigmentation, muscle strength, and mini-mental examination. BMI will be calculated once for the initial Day-1 examination.

Participants will be instructed how to fill out an on-line questionnaire regarding symptoms of adrenal insufficiency. Participants will complete a Cushing's syndrome questionnaire. The questionnaire will ask the participant “In the last few days, how much have you been bothered by the following symptoms? For each symptom, choose the answer that best matches your experience.” The symptoms include Brain fog, Trouble concentrating, Trouble sleeping, Swelling, or bloating, Anxiety, Depression, and Irritability, and the answers are “Not at all, Slightly, Moderately, Extremely and Very”.

### Biospecimen Evaluations

Screening, safety, and efficacy measurements include CBC with differential, liver panel, GGT, ACTH, cortisol, androstenedione, 11-oxygenated androgens (11KT and 11OHΔ4A), 17-OH progesterone, dehydroepiandrosterone (DHEA), progesterone, testosterone, prolactin, IGF-1, growth hormone, acute care panel, POMC-related paptides, frustosamine, insulin, LH, FSH, testosterone (men), estradiol (women), renin activity, aldosterone and pharmacokinetics of Compound 1.

The total blood drawn over the 16-28 days of the study (including variable time for screening), is 279 mL.

Urine samples (cortisol, creatinine, potassium, sodium, volume): Collect urine for 24 hours starting at approximately 0800 on the study days.

### Correlative Studies for Research/Pharmacokinetic Studies

Plasma samples will be collected for measurement of plasma concentration of study drug as specified in the SOA (Table 1). Samples will be collected for PK assessment on both Day 1 and Day 10 at predose and 0.25, 0.5, 1.0, 1.5, 2, 8, and 18 hours after dose administration. PK sampling times in the first 120 minutes should be within ±5 minutes of specified time, thereafter a window of ±30 minutes is allowed. Single PK samples will be collected on Day 11 and Day 12 (approximately 24 and 48 hours after the last dose administration on Day 10, respectively) and on Day 14.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating adrenocorticotropic hormone (ACTH)-dependent Cushing's syndrome in a human comprising orally administering once daily about 80 mg to about 160 mg of a compound having the structure of Compound 1, or a pharmaceutically acceptable salt thereof:

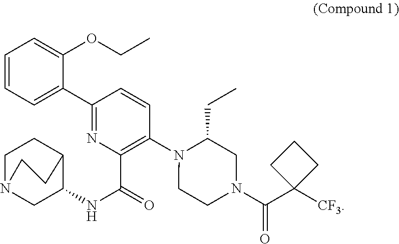 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

2. The method of claim 1, wherein the human with ACTH-dependent Cushing's syndrome comprises a pituitary adenoma.

3. The method of claim 1, wherein the human with ACTH-dependent Cushing's syndrome comprises an ectopic ACTH-secreting tumor.

4. The method of claim 3, wherein the ectopic ACTH-secreting tumor is in the lungs, pancreas, thyroid, thymus gland, intestines, adrenal gland, paraganglia, or combinations thereof.

5. The method of any one of claims 1-4, wherein treating ACTH-dependent Cushing's syndrome comprises lowering serum cortisol levels, lowering urine cortisol levels, or both.

6. The method of claim 5, wherein treating ACTH-dependent Cushing's syndrome comprises lowering cortisol levels in the human by at least 10% from baseline.

7. The method of any one of claims 1-6, wherein treating ACTH-dependent Cushing's syndrome comprises lowering levels of serum cortisol, androstenedione, or combinations thereof.

8. The method of claim 7, wherein treating ACTH-dependent Cushing's syndrome comprises lowering levels of serum cortisol, androstenedione, or combinations thereof, by at least 10% from baseline.

9. The method of any one of claims 1-6, wherein treating ACTH-dependent Cushing's syndrome comprises reducing levels of cortisol, androstenedione (A4), 17-hydroxyprogesterone (17-OHP), aldosterone, dehydroepiandrosterone sulfate (DHEAS), dehydroepiandrosterone (DHEA), or combinations thereof.

10. The method of claim 9, wherein treating ACTH-dependent Cushing's syndrome comprises reducing levels of cortisol, androstenedione (A4), 17-hydroxyprogesterone (17-OHP), aldosterone, dehydroepiandrosterone sulfate (DHEAS), dehydroepiandrosterone (DHEA), or combinations thereof, by at least 10% from baseline.

11. The method of any one of claims 1-10, wherein treating ACTH-dependent Cushing's syndrome comprises delaying or eliminating the need for removal of one or both adrenal glands.

12. The method of any one of claims 1-11, wherein about 80 mg, about 120 mg, or about 160 mg of Compound 1, or a pharmaceutically acceptable salt thereof, is administered.

13. The method of any one of claims 1-11, wherein about 80 mg of Compound 1, or a pharmaceutically acceptable salt thereof, is administered.

14. The method of any one of claims 1-11, wherein about 120 mg of Compound 1, or a pharmaceutically acceptable salt thereof, is administered.

15. The method of any one of claims 1-11, wherein about 160 mg of Compound 1, or a pharmaceutically acceptable salt thereof, is administered.

16. The method of any one of claims 1-15, wherein Compound 1, or a pharmaceutically acceptable salt thereof, is administered once daily in the evening.
